(19) United States
(12) Patent Application Publication
(10) Pub. No.: US 2020/0294634 A1
(43) Pub. Date: Sep. 17, 2020

(54) SYSTEM AND METHOD FOR PROVIDING AND ANALYZING PERSONALIZED PATIENT MEDICAL HISTORY

(71) Applicant: Griffin Katz, New York, NY (US)

(72) Inventor: Griffin Katz, New York, NY (US)

(21) Appl. No.: 16/389,564

(22) Filed: Apr. 19, 2019

Related U.S. Application Data
(60) Provisional application No. 62/816,401, filed on Mar. 11, 2019.
(60) Provisional application No. 62/816,400, filed on Mar. 11, 2019.

(51) Int. Cl. G16H 10/60; G16H 50/20; G16H 50/30

(52) U.S. Cl. CPC G16H 10/60; G16H 80/00; G16H 50/30; G16H 50/20

(57) ABSTRACT

An information providing system is described. The information providing system includes a centralized data system having access to patient medical history information from more than one original source of information. The access is referenced by a patient identifier. A printed pattern represents a coded machine-readable indicia. The coded machine-readable indicia has encrypted therein a reference to the centralized data system. An electronic device is configured to capture the coded machine-readable indicia and send the data relating to the patient identifier to the centralized data system.

### REFERENCE TO PRIORITY APPLICATIONS

This application is a nonprovisional patent application claiming priority to U.S. Provisional patent Application No. 62/816,400, entitled IMAGE CAPTURE OF PATIENT'S PERSONALIZED, READABLE INDICIA (QR-CODE) CONTAINING ALL PAST MEDICAL FINANCIAL DATA CREATING ECOSYSTEM (DATABASE) TO CAPTURE AND ANALYZE FINANCIAL DATA ACROSS POPULATIONS, filed on Mar. 11, 2019, to Inventor Griffin Katz. This application also claims priority to Provisional Application No. 62/816,401, entitled MEDICAL DATA TRANSFERRED VIA IMAGE CAPTURE OF ENCRYPTED, READABLE INDICIA (QR-CODE) BY HEALTHCARE PROFESSIONALS TO SECURELY ACCESS PATIENT'S MEDICAL HISTORY, filed on Mar. 11, 2019, to Inventor Griffin Katz. Both of the priority Provisional Applications are herein incorporated by reference herein in their entirety.

### BACKGROUND

In the conventional healthcare field today, a patient's complete past medical history may not be readily available to themselves or to various healthcare providers. Similarly, a healthcare provider may not have access to a patient's current prescriptions unless told to the healthcare provider by the patient.

For example, currently if I get hurt in Alabama, but live in New York, the healthcare providers in the state of Alabama have no access to my previous medical records or my past medical history. This is especially important in the case of an acute emergency where the patient may be unconscious or otherwise incapacitated to be able to provides information themselves. Furthermore, if that same patient from New York comes in unconscious to a hospital in Alabama, the healthcare professionals have no prior, or current knowledge of that patient. This is a significant issue especially when healthcare costs in the U.S. are so high. If the healthcare provider were able to see the patient's basic medical history, before the patient's consult, healthcare providers would be able to make more informed and directed choices when treating the patient.

For a patient whose name is not in a hospital database system, the daunting task of collecting every bit of data from scratch is a burdensome, time-wasting task for the hospital. Similar to the previous mentioned scenario, if a person from New York were to get hurt in Connecticut (just a 30 minute drive away), the Connecticut's Electronic Medical Record (EMR) system has no record of the person from New York.

Accordingly, there is a need for a system in which a patient's past medical history can be accessed quickly and on the spot. Having this ability can save wasted costs, quality, and access of healthcare. Thus, there is a need for seamlessly integrating the most basic aspects of a patient, and having the healthcare provider aware of a patient's past medical history, before they even consult the patient. Having this ability would significantly improve the quality and cost of healthcare. There would be no more wasted hours recording past medications, or recording every surgery (down to an ingrown toenail in 8th grade) from the patient or their relatives or by ordering records from other healthcare providers. Thus, there is ultimately a need for a system where everything about the patient will be automatically and readily available to the healthcare professionals via their own smartphone.

### SUMMARY

An exemplary embodiment relates to medical information providing system. The medical information providing system includes a centralized data system having access to patient medical history information from more than one original source of information. The access is referenced by a patient identifier. A printed pattern represents a coded machine-readable indicia. The coded machine-readable indicia has encrypted therein a reference to the centralized data system. An electronic device is configured to capture the coded machine-readable indicia and send the data relating to the patient identifier to the centralized data system.

Another exemplary embodiment relates to a method of providing patient medical history information to a mobile phone user. The method includes receiving, at a central data system, a signal representative of a patient identifier. The signal is initially generated by a mobile phone camera automatically capturing a patient specific QR-code. Further, the method includes retrieving from the centralized data system, the patient medical history information associated with the patient identifier and sending the patient medical history information to the mobile phone.

Yet another exemplary embodiment relates to a system of providing patient medical history information to a mobile phone user. The system includes a means for receiving, at a central data system, a signal representative of a patient identifier, the signal being initially generated by a mobile phone camera automatically capturing a patient specific QR code. The system also includes a means for retrieving from the centralized data system, the patient medical history information and prescription information associated with the patient identifier and a means for sending the information to the mobile phone.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a depiction of a QR-Code and depicting a user's access to various information.

FIG. 2 is a depiction of steps for enabling an information system in accordance with exemplary embodiments.

FIG. 3 is a generalized depiction of a system in accordance with exemplary embodiments.

### DETAILED DESCRIPTION

In the following detailed description, reference is made to the accompanying drawings, which form a part thereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

In accordance with an exemplary embodiment, a system and method for Healthcare Professionals to have quick and readily available access to a patient's past medical history, other M.D. visits, current prescriptions, etc. provides numerous advantages to the patient and healthcare provider. A machine-readable indicia which is coupled to or with the patient, such as but not limited to a QR-code, with the patient's past medical history, can save wasted costs, quality, and access to healthcare. In an exemplary embodiment, everything about a patient can be readily uploaded to a healthcare professional's smartphone via capturing a QR-code, or other machine readable indicia, associated with a patient and coupled to a patient or carried with a patient. Currently, doctors, residents, and other healthcare provider personnel all have smartphones which they are able to take pictures of a patient's wounds, unknowns, etc. that get automatically uploaded to their EMR of choice. Thus, in accordance with an exemplary embodiment it would be highly advantageous to have a patient's past medical history available to a healthcare provider in an instant via capturing a QR-code with their smartphone or camera-enabled mobile phone.

The Exemplary embodiments herein described solve the problem of Healthcare providers having to access some or all of a patient's medical history by verbally consulting with the patient. By providing Healthcare professionals a glimpse into a patient, before they even have a consultation via accessing the patient's medical data via capturing an associated QR-code, allows physicians and other healthcare providers to make more informed decisions about the patient's care. Additionally, it saves the extraordinary amount of time and resources that it takes for hospital administrators to gather a patient's past medical history, thereby decreasing administrative costs. Also, via the image capture of a QR-code associated with a patient, the patient's data can be auto-uploaded into the hospital's EMR of choice, providing a more seamless care.

Medical organizations generally need at least five types of data to aid in optimizing costs and care. The types of data that are needed are (1) claims data, (2) laboratory data, (3) imaging data, (4) pharmacy data, and (5) clinical data. This data is critical to identifying quality gaps, where the costs and excessive costs are, and performance of the physicians and other providers. Thus, in order to improve care while balancing costs, access to the five data types is necessary. Organizations must have access to claims data that are updated on a periodic, preferably monthly basis. Ideally, the claims data would be available in real-time, which is available in some organizations, however less frequent periodic data can still be used for clinical care and assessments of quality of care and costs. Laboratory data is critical to clinical care. This data should be available electronically on a real-time basis for providers using appropriate Electronic Health Records (EHRs). Organizations further need real0time access to imaging studies and reports. These data are. Electronic and should be available in real-time such that clinical management has access to all imaging from all facilities available immediately after the tests are performed. This requires overcoming much of the interoperability problem between various health care organizations and their electronic records. Further still, real-time pharmacy data, knowing what drugs a patient is taking and whether they are refilling their prescriptions, are necessary to help understand utilization, adherence, cost, and other aspects of care. Finally, organizations need data on admissions and discharges from hospitals, skilled nursing facilities, and other institutions. This may currently be the weakest of the available data, as it is often not shared at all, or shared in a lackadaisical manner that prevents timely action. There are economic incentives to not share this information between organizations and many EHRs are not yet interoperable, thereby preventing the information from being shared.

Once the information necessary for improving care while balancing costs, is more available then what is needed is to provide this information readily to physicians, nurse practitioners, and other front-line providers about the status of their patients. It is important that healthcare providers know about a patient's recent ER visits, hospitilizations, and other medical interventions. It is also vital to know in real-time any new laboratory, and imaging results. This information needs to be readily available to a clinician during a patient encounter. Physicians, for example, need to have immediate and simplified access to the laboratory, imaging, pharmacy, and other data readily at hand for immediate use during a visit to make informed clinical decisions.

Another source of data that is currently underutilized is the data that may be available to clinicians collected between patient visits via wearables and the like. Conventionally there may be, on average, 30,000 minutes between visits. This is commonly a missed opportunity to collect patient data (from, e.g. wearable sensors, and the like) during this time, leading to better treatment and more advantages in optimizing cost and care.

With better access to data, primary care physicians can become competent and confident generalists that can handle routine problems. It has been seen that about 80% of the time, the generalist already knows what the specialist is going to recommend. Getting a consultation from a specialist does not necessarily enhance patient care, thus the generalist can and should be managing the patient, especially as the generalist has ready access to the necessary data.

Thus, if such data is available to the healthcare provider in an efficient manner, better outcomes can be achieved by the healthcare provider during an encounter.

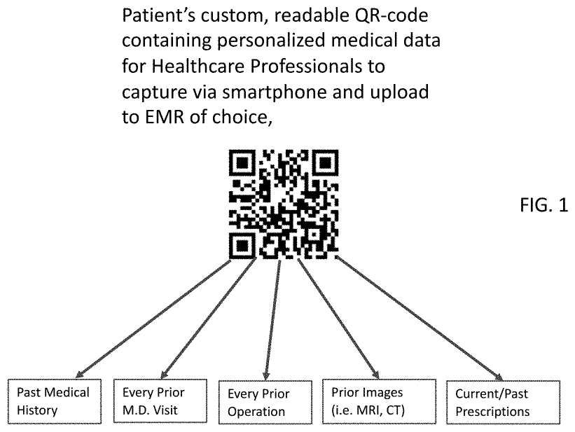

Referring now to FIG. 1, an exemplary QR-code which is customized for an individual patient and associated therewith, is shown. The QR-code can be read by any of a variety of readers including specialized QR-code readers, QR-code reading apps, and smartphone cameras which include software which automatically identifies a QR-code in the field of view of the camera and executes instructions associated with the QR-code. Once a QR-code is read, instructions are automatically carried out which are encrypted in the QR-code to access a patient's medical information. This medical information may include, but is not limited to the patient's Past Medical History, Every Prior Doctor (M.D.) Visit, Every Prior Operation (i.e. MRI, CT), Current and Past Prescriptions, etc.

Conventionally, France requires their 65 million citizens to have a “carte vitale” or vital card which contains all of a patient's medical data on a SIM-card. The “carte vitale” was one of the best ideas to come from the French, however, when it came to actual use among the French, it was as useless as a business card. Many doctors do not have “special-SIM-readers” and the use of SIM-card technology is outdated. The exemplary embodiments described herein are unlike any other, in that it harnesses QR-codes, which can be read by any smartphone (iPhone or Android) in a simple and expedient manner. Furthermore, the patient's medical data is instantly populated and does not require any “special reader,” and the cost of printing QR-codes or other printed machine-readable indicia is negligible. Additionally, no special equipment is required by the patient or other accessor of the medical records since virtually all patients and other persons accessing the records (healthcare personnel, insurers, administrators, etc.) have access to and are likely carrying a mobile phone with a camera. In fact, healthcare organizations today are transforming to digital medicine, some self-imposed and some required by the Affordable Care Act. Now, almost every hospital/university is mandating a basic requirement to have all doctors, nurses, techs, etc. to have a smartphone while on duty.

A machine-readable indicia, such as a QR-code with the patient's past medical history, can save wasted costs, increase quality of care, and provide instant access to healthcare. By seamlessly integrating the most basic aspects of a patient, and having the healthcare provider aware of a patient's past medical history, before they even consult the patient, significantly improves the quality and cost of healthcare. There will be no more wasted hours recording from the patient past medications, or every surgery (down to an ingrown toenail in 8th grade). Everything about the patient will be readily uploaded to the healthcare professionals smartphone via capturing the QR-code.

Also, beyond a patient's past medical history, including for example, every doctor they've ever seen, every doctor they've been referred to, every injection, every operation, every X-ray/MM/CT scan performed, every diagnostic test, and every medication they've ever taken, other information can be included. For example, it may be beneficial to include with the patient record how much every medical doctor has billed the patient, how much insurance paid for a particular visit, and how much the patient paid out of pocket, can also be incorporated into the QR-code.

In accordance with an exemplary embodiment, a patient using the described system uses it in the following exemplary manner. The patient gets a QR-code specific to their current and past medical history (including all PMH, Current Rx's, Referrals, Injections, Operations, Images, Diagnostic Tests, etc.). The patient keeps their personalized QR-code on their person at all times. For example this may be on any of a driver's license, an identification card, an insurance card, a neck chain, a bracelet, a business card, office stationary, other cards, door hangers, presentation folders, bookmarks, office supplies, magnets, labels, stickers, and paper. In the event of getting minorly sick, or in the case of a traumatic emergency, the patient's QR-code will be on their person available to be scanned by the treating healthcare professional. The healthcare professional can review, assess, and decide on treatment plans before even entering the consult with the patient (given that they have all of the patient's medical history before walking into the examining room). The healthcare professional is then able to store the patient's medical data, securely in the EMR (of the choice of the physician), consult with other physicians (now that the patient's medical history is fully in the “system”), and come up with the best possible treatment plan. Additionally, the healthcare provider can upload the patient's medical data to the EMR being used by the physician/hospital. This allows for cross-communication between providers in a system, to deliver higher quality care, in an efficient, and seamless manner.

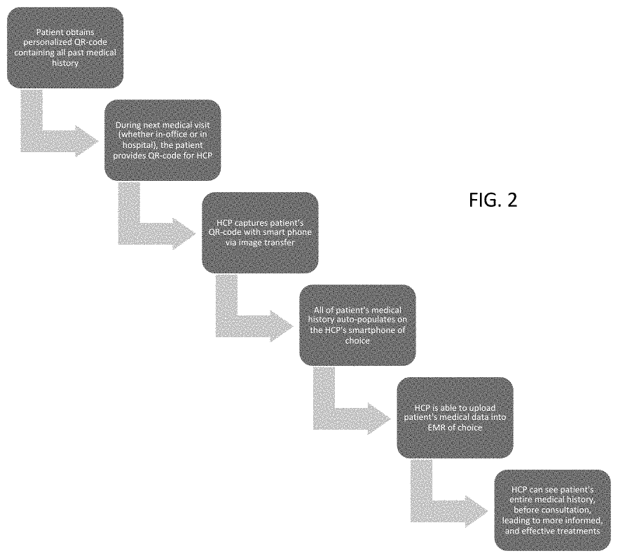

Referring now to FIG. 2, a process is shown in which a patient obtains a personalized QR-code that is associated with records in a central data system that contains all of a patient's past medical history. During the next medical visit of the patient (whether in-office or in-hospital), the patient provides the QR-code to the health care provider (HCP). The HCP captures the patient's QR-code with a smart phone via an image transfer (capture). From the image recognition and capture, all of the patient's medical history auto-populates on the HCP's smartphone. During or after treatment, the HCP is able to upload a patient's medical data into the EMR of choice. The next time the patient is seen by an HCP, the HCP can access and see the patient's entire medical history, before consultation with the patient, leading to a more informed and effective treatment.

A person normally sees their Primary Care Physician or Internist for an annual checkup. During that time, the PCP or Internist writes up the patients medical history (as in every appointment), but instead the Dr. “populates & updates” the patient's history and that updated information sticks with the patient. That is, until the next time they encounter themselves in another doctor's office, that they are able to use the QR-code, to show the new physician, about their past medical history. The new physician would simply capture the patient's QR-code, and would be able to see every data point in the patient's past medical history. Then, according to this information, the “new” physician can make a more informed decision about how to treat the patient (given the patient's past medical history).

A QR-code that has a machine-readable indicia that is able to be captured by any Healthcare provider using a smartphone. Almost all doctors/hospitals have their Healthcare provider's phones integrated into the EMR of choice. Therefore, building off the current concept that any picture a doctor takes gets “auto-uploaded” into a patient's chart, the same concept can be applied to the patient's QR-code, that is all of the patient's information during their encounter with the HCPO can be uploaded to the chosen EMR using the patient's QR-code.

The machine-readable indicia can be anything from a QR-code, to a simple 2-D bar code, etc. However, the item must be convenient to the patient to have on them at all times. Whether it be a QR-code sticker on the back of their phone, ID, wallet, etc. or a wearable such as, a wristband, bracelet, neck chain, temporary or permanent tattoo, etc. This could provide tremendous benefit to a person in the case they need medical care, outside their state of residence.

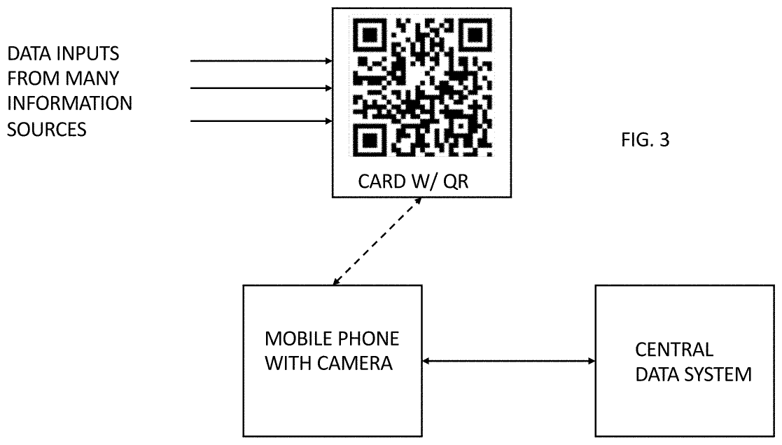

In accordance with an exemplary embodiment as depicted in FIG. 3, a first element of the system is a QR-code displayed on a card or other printed media (such as an insurance or driver's license card, etc.), or additionally can be printed out in the form of a sticker. Encoded within the QR-code is a patient identifier and patient information received from at least one information source, but often aggregated from many sources. The second element of the system is a smartphone, which virtually everyone has in the U.S., especially now most hospitals and clinics require their doctors, nurses, etc. to all have smartphones. The third element of the system is a central data system that can be accessed from a patient identifier coded on the QR-code.

In accordance with an exemplary embodiment, one form of central data system is termed a Vendor Neutral Archive (VNA) of patient data. Vendor Neutral Archives will become the standard in medicine and will be managed in the cloud. Patients may own and control their own data more efficiently. Usage of QR codes, alone, or in conjunction with biometric sensors will increase to control access to this data across multiple stages of the patient journey. This centralization of anonymized data may be accessible by computer vision and artificial intelligence to make precision healthcare more scalable. Aggregation of healthcare data has been a holy grail and promised for the past decades but not delivered. The adoption of secure cloud services is finally delivering this. Anonymizing health data is an important step towards building better algorithms for scalable precision medicine. VNAs archive images and documents in a standard form, standard interface and more easily enable sharing of files. Patients will be able to control access to their own files.

Although the figures or description may describe a specific order of method steps, the order of the steps may differ from what is depicted. In addition, two or more steps may be performed concurrently or with partial concurrence. Such variation will depend on the software and hardware systems chosen and on designer choice. All such variations are within the scope of the disclosure.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of handling patient medical information, comprising:
receiving from a data system patient medical records information, the access referenced by a patient identifier;
encrypting at least some of the patient medical records reformation with the patient identifier into a coded machine-readable indicia;
printing the coded machine-readable indicia on a printable medium; and
providing the printable medium to the patient.

2. The method of claim 1, wherein the coded machine-readable indicia includes a bar code.

3. The method of claim 1, wherein the coded machine-readable indicia includes a QR code.

4. The method of claim 1, wherein the coded machine-readable indicia is read by a mobile phone.

5. The method of claim 1, wherein the coded machine-readable indicia is read by a mobile phone running an app and the app automatically decodes and the patient information from the coded machine-readable indicia and provides at least some of the patient information on a display device.

6. The method of claim 1, wherein the machine-readable indicia is read by a mobile phone running the camera, the camera automatically captures the machine-readable indicia and the app automatically decodes and the patient information from the coded machine-readable indicia and provides at least some of the patient information on a display device.

7. The method of claim 1, wherein the printed pattern is on at least one of a driver's license, an identification card, an insurance card, a neck chain, and a bracelet.

8. A method of providing patient medical history information to a mobile phone user, comprising:
receiving, at a central data system, a signal representative of a patient identifier;
retrieving, from the central data system, patient medical records associated with the patient identifier;
encrypting at least some of the patient medical records information with the patient identifier into a machine-readable QR-code;
printing the machine-readable QR-code on a printable medium; and
providing access to the printable medium by a mobile phone camera to automatically identify the presence of a QR-code and decode information coded on the machine-readable QR-code.

9. The method of providing patient medical history information to a mobile phone user of claim 8, wherein the mobile phone requests a password prior to displaying the patient information.

10. The method of providing patient medical history information to a mobile phone user of claim 8, wherein the machine-readable QR-code includes more than one QR-code.

11. The method of providing patient medical history information to a mobile phone user of claim 8, wherein the machine-readable QR-code includes emergency patient information coded therein.

12. The method of providing patient medical history information to a mobile phone user of claim 8, wherein the machine-readable indicia is read by a mobile phone running an app and the app automatically connects to a central data system and sends a representation of the patient identifier and the patient information thereto.

13. The method of providing patient medical history information to a mobile phone user claim 8, wherein the machine-readable indicia is read by a mobile phone running the camera, the camera automatically capturing the machine-readable QR-code and automatically connecting to a central data system and sends a representation of the patient identifier thereto.

14. The method of providing patient medical history information to a mobile phone user of claim 8, wherein the printed QR-code is on at least one of a driver's license, an identification card, an insurance card, a neck chain, and a bracelet.

15. The method of providing patient medical history information to a mobile phone user of claim 8, wherein a central data system is configured to store prior imaging data or test data of the patient and medical treatment history of the patient and the QR-code includes a reference to where those prior imaging or test data are stored.

16. The method of providing patient medical history information to a mobile phone user of claim 8, wherein the QR-code is configured to provide patient treatment records and prescription records to aid in treatment of the patient.

17. A system of providing patient medical history information to a mobile phone user, comprising:
a means for receiving, at a central data system, a signal representative of a patient identifier, the signal being initially generated by a mobile phone camera automatically capturing a patient specific QR code;
a means for retrieving from the centralized data system, the patient medical history information and prescription information associated with the patient identifier; and
a means for sending the information to the mobile phone.

18. The system of providing patient medical history information to a mobile phone user of claim 17, wherein the machine-readable indicia is read by a mobile phone running the camera, the camera automatically captures the machine-readable indicia and automatically connects to the central data system and sends a representation of the patient identifier thereto.

19. The system of providing patient medical history information to a mobile phone user of claim 17, wherein the central data system is configured to store the medical history information, prescription information and testing and image information of the patient.

20. The method of providing patient medical history information to a mobile phone user of claim 8, wherein the central data system is configured to provide medical\treatment information prescription information to aid in treatment of the patient.
